(19) United States
(12) Patent Application Publication
(10) Pub. No.: US 2021/0128102 A1
(43) Pub. Date: May 6, 2021

(54) ULTRASOUND DIAGNOSTIC APPARATUS AND CONTROL METHOD OF ULTRASOUND DIAGNOSTIC APPARATUS

(71) Applicant: FUJIFILM Corporation, Tokyo (JP)

(72) Inventor: Mitsuru NEGISHI, Kanagawa (JP)

(73) Assignee: FUJIFILM Corporation, Tokyo (JP)

(21) Appl. No.: 17/147,524

(22) Filed: Jan. 13, 2021

Related U.S. Application Data
(63) Continuation of application No. PCT/JP2019/032547, filed on Aug. 21, 2019.

(30) Foreign Application Priority Data
Sep. 4, 2018 (JP) 2018-165277

(51) Int. Cl. A61B 8/08; A61B 8/14; A61B 34/10; A61B 8/00; G16H 30/20; G16H 30/40; G16H 20/40; G16H 50/50; G16H 40/63; G09B 23/28

(52) U.S. Cl. CPC A61B 8/0833; A61B 8/14; A61B 34/10; A61B 8/463; G16H 30/20; A61B 2034/2063; G16H 20/40; G16H 50/50; G16H 40/63; G09B 23/286; A61B 2034/107; G16H 30/40

(57) ABSTRACT

An ultrasound diagnostic apparatus 1 includes an ultrasound probe 18; an image acquisition unit 8 that performs transmission of an ultrasound beam from the ultrasound probe 18 toward a subject to acquire an ultrasound image; a display unit 7 that displays the acquired ultrasound image; a delineation target detection unit 9 that performs an image analysis on the acquired ultrasound image to detect a foreign matter entered a subcutaneous portion of the subject and a peripheral tissue of the subject consisting of blood vessels or nerves positioned around the foreign matter; a foreign matter removal route setting unit 11 that sets a removal route of the foreign matter to avoid the peripheral tissue on the basis of the detected foreign matter and the detected peripheral tissue; and a foreign matter removal route guide unit 12 that provides, to a user, guidance on the set removal route.

### CROSS-REFERENCE TO RELATED APPLICATIONS

This application is a Continuation of PCT International Application No. PCT/JP2019/032547 filed on Aug. 21, 2019, which claims priority under 35 U.S.C. § 119(a) to Japanese Patent Application No. 2018-165277 filed on Sep. 4, 2018. The above application is hereby expressly incorporated by reference, in its entirety, into the present application.

### BACKGROUND OF THE INVENTION

### 1. Field of the Invention

The invention relates to an ultrasound diagnostic apparatus and a control method of the ultrasound diagnostic apparatus, and particularly to an ultrasound diagnostic apparatus and a control method of the ultrasound diagnostic apparatus which are used to remove a foreign matter that has entered a subcutaneous portion of a subject.

### 2. Description of the Related Art

In the related art, an ultrasound diagnostic apparatus has been known as an apparatus for obtaining an image of the inside of a subject. The ultrasound diagnostic apparatus generally comprises an ultrasound probe comprising a transducer array in which a plurality of elements are arranged. In a state where the ultrasound probe is in contact with a body surface of the subject, an ultrasound beam is transmitted toward the subject from the transducer array and an ultrasound echo from the subject is received by the transducer array so that element data is acquired. Further, the ultrasound diagnostic apparatus electrically processes the obtained element data to generate an ultrasound image of the corresponding site of the subject.

Removing a foreign matter while observing the foreign matter entered the subcutaneous portion of the subject is generally performed by using such an ultrasound diagnostic apparatus. In a case where the foreign matter is removed from the subcutaneous portion of the subject in this manner, normally, it is required to grasp the position of the foreign matter by visually observing the ultrasound image and appropriately operate a treatment tool for removing the foreign matter, and therefore specialized knowledge and proficiency are required.

Thus, in order to easily remove the foreign matter, for example, an ultrasound diagnostic apparatus as disclosed in JP2015-204988A has been developed. The ultrasound diagnostic apparatus disclosed in JP2015-204988A acquires a position of a distal end of a treatment tool on the basis of positional information from a position sensor provided on the treatment tool, and displays the position of the distal end of the treatment tool in an ultrasound image in an emphasizing manner to allow the user to grasp the position of the distal end of the treatment tool.

### SUMMARY OF THE INVENTION

However, in the ultrasound diagnostic apparatus disclosed in JP2015-204988A, since it is required for the user to grasp the structure of the foreign matter entered the subcutaneous portion of the subject by visually observing the ultrasound image and decide an appropriate angle or the like of the treatment tool for removing the foreign matter, and the user is still required to have specialized knowledge and proficiency, there is a problem that it is difficult for a user with a low level of proficiency to remove the foreign matter.

The invention has been made in order to solve such a problem in the related art, and an object of the invention is to provide an ultrasound diagnostic apparatus and a control method of the ultrasound diagnostic apparatus which can easily remove a foreign matter regardless of the proficiency of a user.

In order to achieve the object, an ultrasound diagnostic apparatus according to an aspect of the invention includes an ultrasound probe; an image acquisition unit that performs transmission of an ultrasound beam from the ultrasound probe toward a subject to acquire an ultrasound image; a display unit that displays the ultrasound image acquired by the image acquisition unit; a delineation target detection unit that performs an image analysis on the ultrasound image acquired by the image acquisition unit to detect a foreign matter entered a subcutaneous portion of the subject and a peripheral tissue of the subject consisting of blood vessels or nerves positioned around the foreign matter; a foreign matter removal route setting unit that sets a removal route of the foreign matter to avoid the peripheral tissue on the basis of the foreign matter and the peripheral tissue detected by the delineation target detection unit; and a foreign matter removal route guide unit that provides, to a user, guidance on the removal route set by the foreign matter removal route setting unit.

It is preferable that the foreign matter removal route guide unit provides, to the user, guidance on the removal route by superimposing and displaying the removal route set by the foreign matter removal route setting unit on the ultrasound image displayed on the display unit.

It is preferable that the ultrasound diagnostic apparatus further includes a probe operation guide unit that provides, to the user, guidance on an operation of the ultrasound probe such that the foreign matter is delineated on the display unit by a plurality of tomographic images; and a three-dimensional structure acquisition unit that acquires a three-dimensional structure of the foreign matter on the basis of ultrasound images of a plurality of frames representing the plurality of tomographic images of the foreign matter, in which the foreign matter removal route setting unit sets the removal route on the basis of a position of the foreign matter, the three-dimensional structure of the foreign matter, and a position of the peripheral tissue.

Further, the probe operation guide unit may provide, to the user, guidance on the operation of the ultrasound probe such that the foreign matter is delineated by a first tomographic image along a long axis of the foreign matter and a second tomographic image along a short axis of the foreign matter on the display unit, the three-dimensional structure acquisition unit may acquire a length along the short axis of the foreign matter as the three-dimensional structure of the foreign matter from the ultrasound image representing the first tomographic image and the ultrasound image representing the second tomographic image, and the foreign matter removal route setting unit may set the removal route on the basis of the position of the foreign matter, the length along the short axis of the foreign matter, and the position of the peripheral tissue.

Here, in a case where the length along the short axis of the foreign matter acquired by the three-dimensional structure acquisition unit is equal to or less than a predetermined threshold value, the foreign matter removal route setting unit may set the removal route such that the foreign matter avoids the peripheral tissue in the first tomographic image.

Further, in a case where an extension region where the foreign matter extends along a long axis direction is apart from the peripheral tissue in the first tomographic image, the foreign matter removal route setting unit may set the extension region as the removal route, and in a case where the extension region is in contact with the peripheral tissue in the first tomographic image, the foreign matter removal route setting unit may set an inclination region which passes through a distal end portion of the foreign matter and is obtained by tilting the extension region to avoid the peripheral tissue, as the removal route.

In a case where the length along the short axis of the foreign matter acquired by the three-dimensional structure acquisition unit is greater than a predetermined threshold value, the probe operation guide unit may provide, to the user, guidance on the operation of the ultrasound probe such that the foreign matter is delineated by a plurality of tomographic images along a long axis of the foreign matter on the display unit, and the foreign matter removal route setting unit may set the removal route such that the foreign matter avoids the peripheral tissue in the plurality of tomographic images.

Further, in a case where an extension region where the foreign matter extends along a long axis direction is apart from the peripheral tissue in the plurality of tomographic images, the foreign matter removal route setting unit may set the extension region as the removal route, and in a case where the extension region is in contact with the peripheral tissue in the plurality of tomographic images, the foreign matter removal route setting unit may set an inclination region which passes through a distal end portion of the foreign matter and is obtained by tilting the extension region to avoid the peripheral tissue, as the removal route.

The three-dimensional structure acquisition unit may acquire a three-dimensional model of the foreign matter by synthesizing an image of the foreign matter detected by the delineation target detection unit with the plurality of ultrasound images representing the plurality of tomographic images, and the foreign matter removal route setting unit may set the removal route on the basis of the position of the foreign matter, a length along a short axis of the three-dimensional model, and the position of the peripheral tissue.

Here, in a case where the length along the short axis of the three-dimensional model acquired by the three-dimensional structure acquisition unit is equal to or less than a predetermined threshold value, the foreign matter removal route setting unit may set the removal route such that the foreign matter avoids the peripheral tissue in the tomographic image along a long axis of the foreign matter.

Further, in a case where an extension region where the foreign matter extends along a long axis direction is apart from the peripheral tissue in the tomographic image along the long axis of the foreign matter, the foreign matter removal route setting unit may set the extension region as the removal route, and in a case where the extension region is in contact with the peripheral tissue in the tomographic image along the long axis of the foreign matter, the foreign matter removal route setting unit may set an inclination region which passes through a distal end portion of the foreign matter and is obtained by tilting the extension region to avoid the peripheral tissue, as the removal route.

In a case where the length along the short axis of the three-dimensional model acquired by the three-dimensional structure acquisition unit is greater than a predetermined threshold value, the probe operation guide unit may provide, to the user, guidance on the operation of the ultrasound probe such that the foreign matter is delineated by a plurality of tomographic images along a long axis of the foreign matter on the display unit, and the foreign matter removal route setting unit may set the removal route such that the foreign matter avoids the peripheral tissue in the plurality of tomographic images.

Further, in a case where an extension region where the foreign matter extends along a long axis direction is apart from the peripheral tissue in the plurality of tomographic images, the foreign matter removal route setting unit may set the extension region as the removal route, and in a case where the extension region is in contact with the peripheral tissue in the plurality of tomographic images, the foreign matter removal route setting unit may set an inclination region which passes through a distal end portion of the foreign matter and is obtained by tilting the extension region to avoid the peripheral tissue, as the removal route.

The delineation target detection unit may detect a treatment tool for removing the foreign matter, and the foreign matter removal route guide unit may provide guidance to the user to modify a position of the treatment tool in a case where the treatment tool detected by the delineation target detection unit deviates from the removal route.

Further, the ultrasound diagnostic apparatus may further include an emphasizing unit that emphasizes at least one of the foreign matter or the treatment tool detected by the delineation target detection unit and displays the resultant on the display unit.

A control method of an ultrasound diagnostic apparatus according to another aspect of the invention includes performing transmission of an ultrasound beam toward a subject to acquire an ultrasound image; performing an image analysis on the acquired ultrasound image to detect a foreign matter entered a subcutaneous portion of the subject and a peripheral tissue of the subject consisting of blood vessels or nerves positioned around the foreign matter; setting a removal route of the foreign matter to avoid the peripheral tissue on the basis of the detected foreign matter and the detected peripheral tissue; and providing, to a user, guidance on the set removal route.

According to the invention, since the ultrasound diagnostic apparatus includes the delineation target detection unit that performs an image analysis on the ultrasound image acquired by the image acquisition unit to detect the foreign matter entered the subcutaneous portion of the subject and the peripheral tissue of the subject consisting of blood vessels or nerves positioned around the foreign matter; the foreign matter removal route setting unit that sets the removal route of the foreign matter to avoid the peripheral tissue on the basis of the foreign matter and the peripheral tissue detected by the delineation target detection unit; and the foreign matter removal route guide unit that provides, to the user, guidance on the removal route set by the foreign matter removal route setting unit, it is possible to easily remove the foreign matter regardless of the proficiency of a user.

### BRIEF DESCRIPTION OF THE DRAWINGS

FIG. 1 is a block diagram illustrating a configuration of an ultrasound diagnostic apparatus according to a first embodiment of the invention.

FIG. 2 is a block diagram illustrating an internal configuration of a reception circuit in the first embodiment of the invention.

FIG. 3 is a block diagram illustrating an internal configuration of an image generation unit in the first embodiment of the invention.

FIG. 4 is a flowchart illustrating an operation of the ultrasound diagnostic apparatus according to the first embodiment of the invention.

FIG. 5 is a diagram schematically illustrating a long-axis image of a foreign matter.

FIG. 6 is a diagram schematically illustrating a short-axis image of a foreign matter.

FIG. 7 is a flowchart illustrating an operation of setting a removal route of a foreign matter in a case where the length of the foreign matter along a short axis is equal to or less than a threshold value.

FIG. 8 is a diagram schematically illustrating an extension region in contact with a peripheral tissue.

FIG. 9 is a diagram schematically illustrating an inclination region.

FIG. 10 is a diagram illustrating a distance between the inclination region and the peripheral tissue.

FIG. 11 is a diagram schematically illustrating an extension region apart from the peripheral tissue.

FIG. 12 is a flowchart illustrating an operation of providing guidance on the removal route.

FIG. 13 is a flowchart illustrating an operation of setting the removal route of the foreign matter in a case where the length of the foreign matter along the short axis is greater than the threshold value.

FIG. 14 is a diagram schematically illustrating the extension region in contact with the peripheral tissue in a case where an ultrasound probe is moved in a depth direction.

FIG. 15 is a diagram schematically illustrating the inclination region in a case where the ultrasound probe is moved in the depth direction.

FIG. 16 is a diagram schematically illustrating the extension region apart from the peripheral tissue in a case where the ultrasound probe is moved in the depth direction.

FIG. 17 is a flowchart illustrating an operation of an ultrasound diagnostic apparatus according to a second embodiment of the invention.

### DESCRIPTION OF THE PREFERRED EMBODIMENTS

Hereinafter, embodiments of the invention will be described with reference to the accompanying drawings.

### First Embodiment

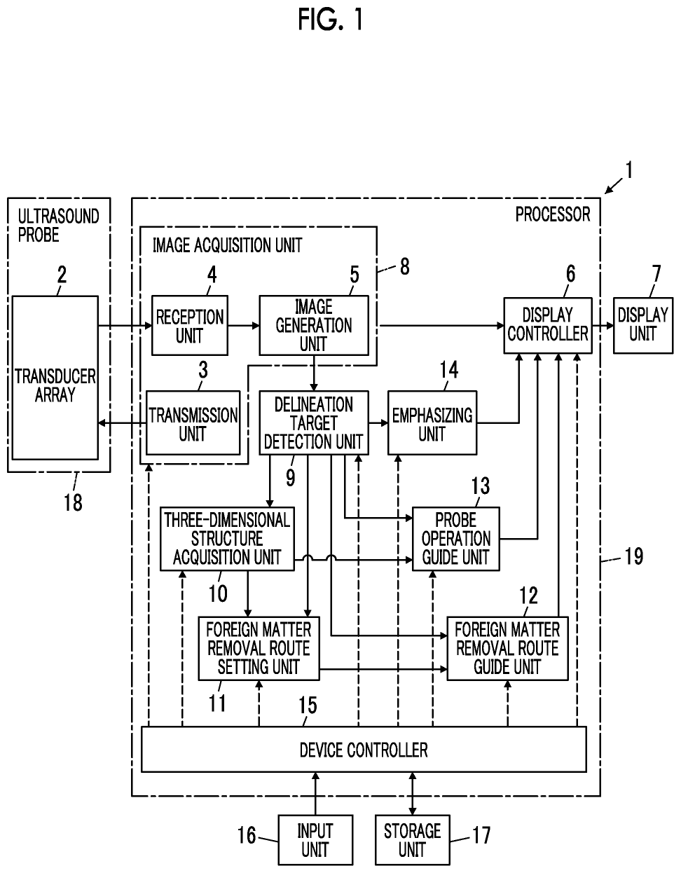

FIG. 1 illustrates a configuration of an ultrasound diagnostic apparatus 1 according to a first embodiment of the invention. As illustrated in FIG. 1, the ultrasound diagnostic apparatus 1 comprises a transducer array 2, and each of a transmission unit 3 and a reception unit 4 is connected to the transducer array 2. An image generation unit 5, a display controller 6, and a display unit 7 are sequentially connected to the reception unit 4. Here, the transmission unit 3, the reception unit 4, and the image generation unit 5 constitute an image acquisition unit 8. A delineation target detection unit 9 is connected to the image generation unit 5, and a three-dimensional structure acquisition unit 10 is connected to the delineation target detection unit 9. A foreign matter removal route setting unit 11 is connected to the delineation target detection unit 9 and the three-dimensional structure acquisition unit 10. A foreign matter removal route guide unit 12 is connected to the delineation target detection unit 9 and the foreign matter removal route setting unit 11. A probe operation guide unit 13 is connected to the delineation target detection unit 9 and the three-dimensional structure acquisition unit 10. An emphasizing unit 14 is connected to the delineation target detection unit 9. In addition, the display controller 6 is connected to the foreign matter removal route guide unit 12, the probe operation guide unit 13, and the emphasizing unit 14.

Further, a device controller 15 is connected to the display controller 6, the image acquisition unit 8, the delineation target detection unit 9, the three-dimensional structure acquisition unit 10, the foreign matter removal route setting unit 11, the foreign matter removal route guide unit 12, the probe operation guide unit 13, and the emphasizing unit 14, and an input unit 16 and a storage unit 17 are connected to the device controller 15. Here, the device controller 15 and the storage unit 17 are connected so as to exchange information bidirectionally.

In addition, the display controller 6, the image acquisition unit 8, the delineation target detection unit 9, the three-dimensional structure acquisition unit 10, the foreign matter removal route setting unit 11, the foreign matter removal route guide unit 12, the probe operation guide unit 13, the emphasizing unit 14, and the device controller 15 constitute a processor 19.

The transducer array 2 of an ultrasound probe 18 illustrated in FIG. 1 has a plurality of transducers arranged in a one-dimensional or two-dimensional manner. According to a drive signal supplied from the transmission unit 3, each of the transducers transmits an ultrasonic wave and receives an ultrasound echo from a subject to output a reception signal. For example, each transducer is configured by forming electrodes at both ends of a piezoelectric body consisting of piezoelectric ceramic represented by lead zirconate titanate (PZT), a polymer piezoelectric element represented by poly vinylidene di fluoride (PVDF), piezoelectric single crystal represented by lead magnesium niobate-lead titanate (PMN-PT), or the like.

The transmission unit 3 of the image acquisition unit 8 includes, for example, a plurality of pulse generators, and the transmission unit 3 adjusts the amount of delay of each drive signal so that ultrasonic waves transmitted from the plurality of transducers of the transducer array 2 form an ultrasound beam on the basis of a transmission delay pattern selected according to a control signal from the device controller 15, and supplies the obtained signals to the plurality of transducers. Thus, in a case where a pulsed or continuous-wave voltage is applied to the electrodes of the plurality of transducers of the transducer array 2, the piezoelectric body expands and contracts to generate pulsed or continuous-wave ultrasonic waves from each transducer. From the combined wave of these ultrasonic waves, an ultrasound beam is formed.

The transmitted ultrasound beam is reflected by a target, for example, a site of the subject, and propagates toward the transducer array 2 of the ultrasound probe 18. The ultrasound echo propagating toward the transducer array 2 in this manner is received by each transducer constituting the transducer array 2. In this case, each transducer constituting the transducer array 2 expands and contracts by receiving the propagating ultrasound echo to generate electrical signals, and outputs the electrical signals to the reception unit 4.

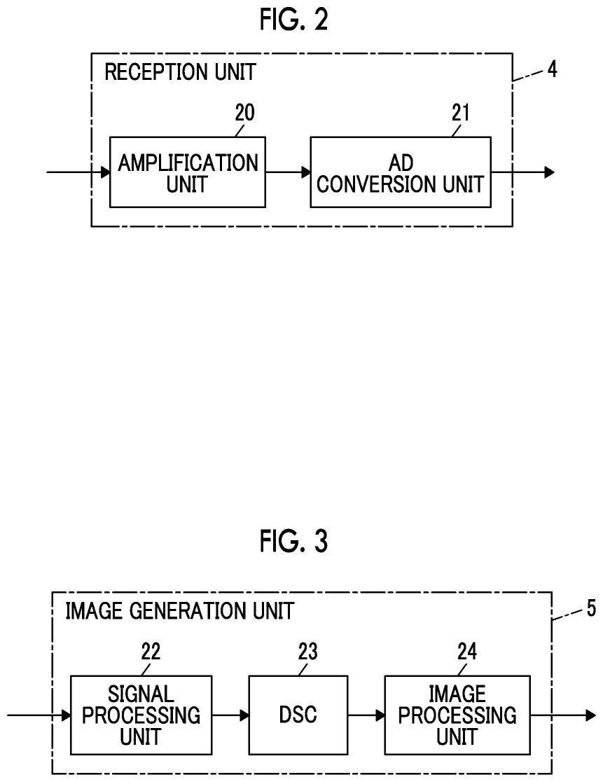

The reception unit 4 of the image acquisition unit 8 processes the signals output from the transducer array 2 according to the control signal from the device controller 15. As illustrated in FIG. 2, the reception unit 4 has a configuration in which an amplification unit 20 and an analog digital (AD) conversion unit 21 are connected in series. The amplification unit 20 amplifies the signals input from each transducer constituting the transducer array 2, and transmits the amplified signals to the AD conversion unit 21. The AD conversion unit 21 converts the signals transmitted from the amplification unit 20 into digitized reception signals, and sends the data to the image generation unit 5 of the image acquisition unit 8.

As illustrated in FIG. 3, the image generation unit 5 of the image acquisition unit 8 has a configuration in which a signal processing unit 22, a digital scan converter (DSC) 23, and an image processing unit 24 are connected in series. The signal processing unit 22 performs reception focusing processing in which addition (phasing addition) is performed by giving delays to respective pieces of the data of the reception signals, on the basis of a reception delay pattern selected according to the control signal from the device controller 15. Through the reception focusing processing, a sound ray signal in which the focus of the ultrasound echo is narrowed to one scanning line is generated. The signal processing unit 22 generates a B-mode image signal, which represents tissues inside the subject, by performing envelope detection processing after correcting the attenuation of the generated sound ray signal which is caused by the propagation distance according to the depth of the reflection position of the ultrasonic wave. The B-mode image signal generated in this manner is output to the DSC 23.

The DSC 23 of the image generation unit 5 generate the ultrasound image by raster-converting on the B-mode image signal into an image signal according to a normal television signal scanning method. The image processing unit 24 of the image generation unit 5 performs various kinds of required image processing, such as brightness correction, gradation correction, sharpness correction, and color correction, on the ultrasound image obtained in the DSC 23, and then outputs the ultrasound image to the display controller 6 and the delineation target detection unit 9.

The delineation target detection unit 9 of the processor 19 performs an image analysis on the ultrasound image acquired from the image acquisition unit 8, and detects, as a delineation target, a foreign matter such as a wood piece, a glass piece, and a metal piece entered the subcutaneous portion of the subject, and a peripheral tissue consisting of blood vessels or nerves positioned around the foreign matter. Further, the delineation target detection unit can detect, as the delineation target, the treatment tool such as forceps and a puncture needle for removing the foreign matter. More specifically, the delineation target detection unit 9 can detect the delineation target, for example, by storing typical pattern data in advance as a template, calculating a pattern data similarity while searching images with the template, and considering that the delineation target is present in a place where the similarity is equal to or greater than the threshold value and is the maximum.

For the calculation of the similarity, in addition to simple template matching, for example, a machine learning method described in Csurka et al.: Visual Categorization with Bags of Keypoints, Proc. of ECCV Workshop on Statistical Learning in Computer Vision, pp. 59-74 (2004) or a general image recognition method using deep learning described in Krizhevsk et al.: ImageNet Classification with Deep Convolutional Neural Networks, Advances in Neural Information Processing Systems 25, pp. 1106-1114 (2012) can be used.

The three-dimensional structure acquisition unit 10 of the processor 19 acquires the three-dimensional structure of the foreign matter on the basis of the ultrasound images of a plurality of frames representing a plurality of tomographic images in which the foreign matter is detected by the delineation target detection unit 9. For example, the three-dimensional structure acquisition unit 10 can measure the length along the long axis of the foreign matter, the length along the short axis of the foreign matter, and the like, as the three-dimensional structure of the foreign matter, on the basis of the tomographic images along the long axis of the foreign matter and the tomographic images along the short axis of the foreign matter.

The foreign matter removal route setting unit 11 of the processor 19 sets the removal route for removing the foreign matter on the basis of the position of the foreign matter and the position of the peripheral tissue detected by the delineation target detection unit 9, and the three-dimensional structure of the foreign matter acquired by the three-dimensional structure acquisition unit 10. For example, the foreign matter removal route setting unit 11 can set the removal route on the basis of the position of the foreign matter, the position of the peripheral tissue, the length along the short axis of the foreign matter, and the like. The setting of the removal route by the foreign matter removal route setting unit 11 will be described in detail later.

The foreign matter removal route guide unit 12 of the processor 19 provides guidance on the removal route set by the foreign matter removal route setting unit 11, to the user. For example, the foreign matter removal route guide unit 12 can provide, to the user, guidance on the removal route by superimposing and displaying the removal route on the ultrasound image displayed on the display unit 7.

The emphasizing unit 14 of the processor 19 emphasizes and displays the delineation target detected by the delineation target detection unit 9 on the display unit 7. For example, the emphasizing unit 14 can display the foreign matter, the peripheral tissue, the treatment tool, and the like in the ultrasound image, on the display unit 7 in different colors. Further, for example, the emphasizing unit 14 can also perform display by so-called shading on the foreign matter, the peripheral tissue, the treatment tool, and the like in the ultrasound image, and can display a contour line representing the contour of the foreign matter, the peripheral tissue, the treatment tool, and the like, on the display unit 7.

The device controller 15 of the processor 19 controls each unit of the ultrasound diagnostic apparatus 1 on the basis of the program recorded in advance in the storage unit 17 or the like and the user's operation through the input unit 16.

The display controller 6 of the processor 19 causes the display unit 7 to display the ultrasound image generated by the image generation unit 5 of the image acquisition unit 8, the removal route set by the foreign matter removal route setting unit 11, the guidance by the foreign matter removal route guide unit 12, and the guidance by the probe operation guide unit 13, under the control of the device controller 15.

The display unit 7 of the ultrasound diagnostic apparatus 1 is for displaying the ultrasound image generated by the image acquisition unit 8, the removal route set by the foreign matter removal route setting unit 11, the guidance by the foreign matter removal route guide unit 12, the guidance by the probe operation guide unit 13, and the like, and includes a display device such as a liquid crystal display (LCD), or an organic electroluminescence (EL) display.

The input unit 16 of the ultrasound diagnostic apparatus 1 is for the user to perform an input operation, and can be configured to comprise a keyboard, a mouse, a trackball, a touchpad, a touch panel, and the like.

The storage unit 17 stores an operation program and the like of the ultrasound diagnostic apparatus 1, and recording media such as a hard disc drive (HDD), a solid state drive (SSD), a flexible disc (FD), a magneto-optical disc (MO disc), a magnetic tape (MT), a random access memory (RAM), a compact disc (CD), a digital versatile disc (DVD), a secure digital card (SD card), and a universal serial bus memory (USB memory), or a server can be used.

The processor 19 having the display controller 6, the image acquisition unit 8, the delineation target detection unit 9, the three-dimensional structure acquisition unit 10, the foreign matter removal route setting unit 11, the foreign matter removal route guide unit 12, the probe operation guide unit 13, the emphasizing unit 14, and the device controller 15 is configured by a central processing unit (CPU) and a control program causing the CPU to execute various kinds of processing, but the processor 19 may be configured by using a field programmable gate array (FPGA), a digital signal processor (DSP), an application specific integrated circuit (ASIC), a graphics processing unit (GPU), or other integrated circuits (IC) or may be configured by a combination thereof.

Similarly, the display controller 6, the image acquisition unit 8, the delineation target detection unit 9, the three-dimensional structure acquisition unit 10, the foreign matter removal route setting unit 11, the foreign matter removal route guide unit 12, the probe operation guide unit 13, the emphasizing unit 14, and the device controller 15 of the processor 19 can be configured by being integrated partially or entirely into one CPU.

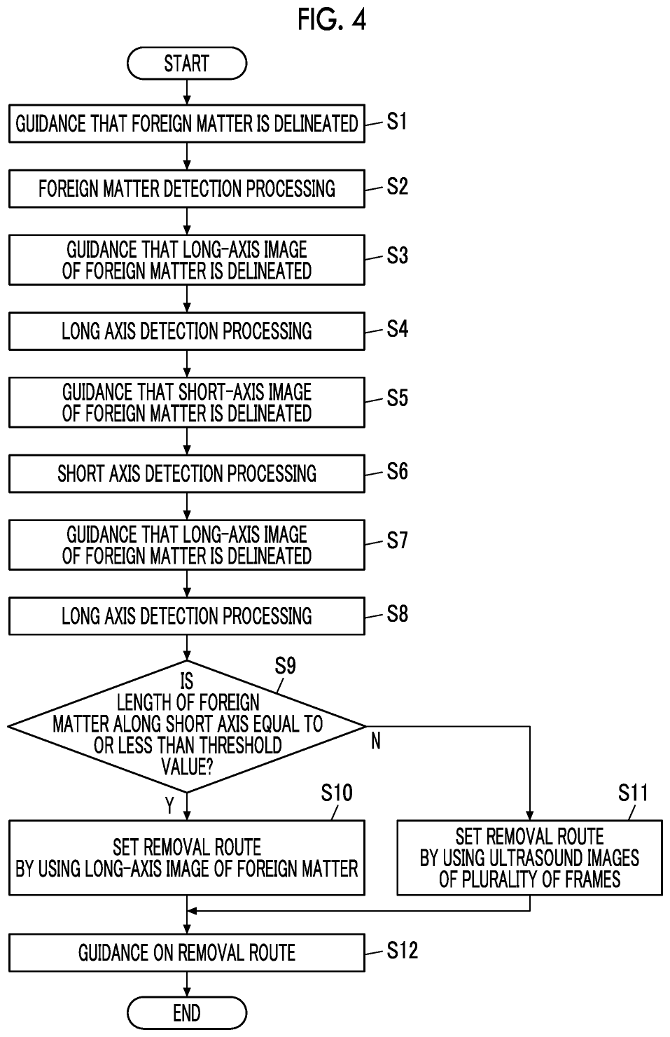

Next, the operation of the ultrasound diagnostic apparatus 1 in the first embodiment will be described in detail using the flowchart illustrated in FIG. 4. In the following operation description, it is assumed that the ultrasound images are sequentially and continuously acquired by the image acquisition unit 8 of the processor 19 to be displayed on the display unit 7, and the ultrasound probe 18 is moved by the user for the observation of the foreign matter entered the subcutaneous portion of the subject.

In Step S1, the probe operation guide unit 13 provides guidance that the foreign matter is delineated on the display unit 7, to the user. Although not illustrated, the probe operation guide unit 13 displays the text, image, and the like representing that the foreign matter is delineated on the display unit 7 to provide guidance to the user, under the control of the device controller 15. The user moves the ultrasound probe 18 according to the guidance by the probe operation guide unit 13. In this case, the image acquisition unit 8 acquires the ultrasound images of a plurality of frames sequentially and continuously.

Next, in Step S2, the delineation target detection unit 9 performs an image analysis on each of the ultrasound images of the plurality of frames sequentially acquired by the image acquisition unit 8, and performs processing of detecting the foreign matter as the delineation target. In this case, the delineation target detection unit 9 performs the processing of detecting the foreign matter on each of the ultrasound images sequentially acquired by the image acquisition unit 8 until the foreign matter is detected. In a case where the foreign matter is detected in Step S2, the processing proceeds to Step S3.

In Step S3, the probe operation guide unit 13 provides guidance that a first tomographic image of the foreign matter along the long axis, that is, the long-axis image of the foreign matter is delineated on the display unit 7, to the user. The user moves the ultrasound probe 18 according to the guidance by the probe operation guide unit 13.

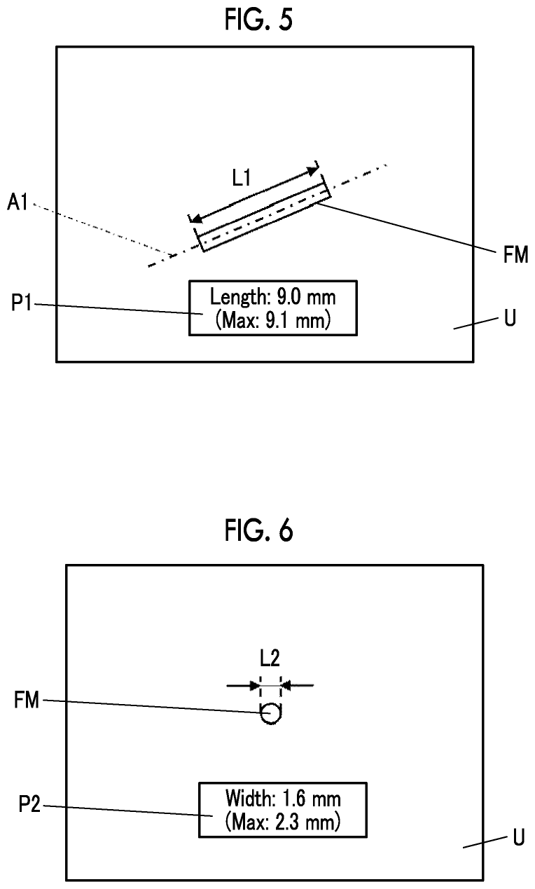

In subsequent Step S4, the three-dimensional structure acquisition unit 10 performs an image analysis on each of the ultrasound images of the plurality of frames sequentially acquired by the image acquisition unit 8, and performs processing of detecting the long axis of the foreign matter included in the ultrasound image. In this case, for example, as illustrated in FIG. 5, first, the three-dimensional structure acquisition unit 10 detects a principal axis of inertia A1 of a foreign matter FM in an ultrasound image U, and measures a length L1 of the foreign matter FM along the detected principal axis of inertia A1. The three-dimensional structure acquisition unit 10 performs the detection of the principal axis of inertia A1 and the measurement of the length L1 on each of the ultrasound images U sequentially acquired by the image acquisition unit 8. Further, for example, in the ultrasound images U of the plurality of frames continuous in time series, in a case where a certain period of time has elapsed while the length L1 of the foreign matter FM along the principal axis of inertia A1 is not changed in a state of being maximized, the three-dimensional structure acquisition unit 10 decides the principal axis of inertia A1 in this case as the long axis of the foreign matter FM, and decides the ultrasound image U including the principal axis of inertia A1 as the long-axis image of the foreign matter FM.

In this case, for example, the three-dimensional structure acquisition unit 10 can hold the maximum value of the length L1 measured in the processing of detecting the long axis of the foreign matter FM, and can display the maximum value and the current value of the length L1 on the display unit 7. In the example illustrated in FIG. 5, a measured value panel P1 including the text representing the maximum value and the current value of the length L1 is superimposed and displayed on the ultrasound image U.

The emphasizing unit 14 can emphasize the foreign matter FM in the ultrasound image U and display the resultant on the display unit 7. In this manner, the user can easily grasp the foreign matter FM in the ultrasound image U by visual observation.

Next, in Step S5, the probe operation guide unit 13 provides guidance that a second tomographic image of the foreign matter FM along the short axis, that is, the short-axis image of the foreign matter FM is delineated on the display unit 7, to the user under the control of the device controller 15. For example, the probe operation guide unit 13 provides, to the user, guidance that the short-axis image of the foreign matter FM is delineated by rotating the ultrasound probe 18 by 90 degrees from the state in which the long-axis image of the foreign matter FM is delineated in the ultrasound image U as illustrated in FIG. 5. In this manner, the user rotates the ultrasound probe 18 according to the guidance by the probe operation guide unit 13.

In subsequent Step S6, the three-dimensional structure acquisition unit 10 performs an image analysis on each of the ultrasound images U of the plurality of frames sequentially acquired by the image acquisition unit 8, and performs processing of detecting the short axis of the foreign matter FM included in the ultrasound image U. In this case, for example, as illustrated in FIG. 6, the three-dimensional structure acquisition unit 10 measures a length L2 of the foreign matter FM along a horizontal direction of the ultrasound image U. The three-dimensional structure acquisition unit 10 measures the length L2 of the foreign matter FM in each of the ultrasound images U sequentially acquired by the image acquisition unit 8, and in a case where a certain period of time has elapsed while the length L2 of the foreign matter FM is not changed in a state of being maximized in the ultrasound images U of the plurality of frames continuous in time series, the three-dimensional structure acquisition unit 10 decides and holds the length L2 as the length along the short axis of the foreign matter FM. Further, the three-dimensional structure acquisition unit 10 decides the ultrasound image U in this case as the short-axis image of the foreign matter FM.

In this case, for example, the three-dimensional structure acquisition unit 10 can hold the maximum value of the length L2 measured in the processing of detecting the short axis of the foreign matter FM, and can display the maximum value and the current value of the length L2 on the display unit 7. In the example illustrated in FIG. 6, a measured value panel P2 including the text representing the maximum value and the current value of the length L2 is superimposed and displayed on the ultrasound image U.

In Step S7, the probe operation guide unit 13 provides, to the user, guidance that the long-axis image of the foreign matter FM is delineated on the display unit 7, in order to display the ultrasound image for setting the removal route of the foreign matter FM on the display unit 7. In this case, for example, the probe operation guide unit 13 provides, to the user, guidance that the long-axis image of the foreign matter FM is delineated by rotating the ultrasound probe 18 by 90 degrees from the state in which the short-axis image of the foreign matter FM is delineated in the ultrasound image U as illustrated in FIG. 6. In this manner, the user rotates the ultrasound probe 18 according to the guidance by the probe operation guide unit 13.

In Step S8, the three-dimensional structure acquisition unit 10 performs processing of detecting the long axis of the foreign matter FM included in the ultrasound image U, as in Step S4. That is, the three-dimensional structure acquisition unit 10 performs the detection of the principal axis of inertia A1 of the foreign matter FM and the measurement of the length L1 of the foreign matter FM along the detected principal axis of inertia A1 on each of the ultrasound images U sequentially acquired by the image acquisition unit 8, and in a case where a certain period of time has elapsed while the length L1 of the foreign matter FM along the principal axis of inertia A1 is not changed in a state of being maximized in the ultrasound images U of the plurality of frames continuous in time series, the three-dimensional structure acquisition unit 10 decides the principal axis of inertia A1 in this case as the long axis of the foreign matter FM, and decides the ultrasound image U including the principal axis of inertia A1 as the long-axis image of the foreign matter FM.

In Step S9, the foreign matter removal route setting unit 11 determines whether the length of the foreign matter FM along the short axis, which is measured in Step S6, is equal to or less than a predetermined threshold value. Here, the threshold value can be set in a range of 8 mm to 10 mm, for example. In a case where the length of the foreign matter FM along the short axis is equal to or less than the threshold value, the processing proceeds to Step S10.

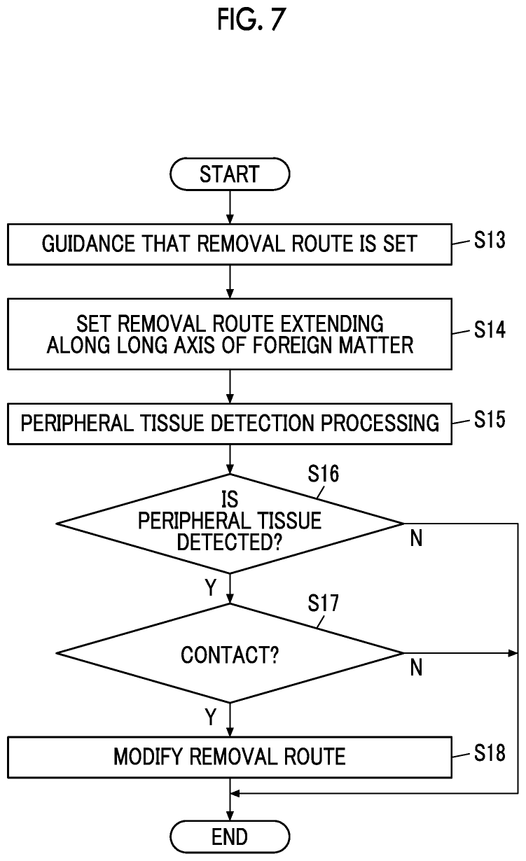

In Step S10, the foreign matter removal route setting unit 11 sets the removal route of the foreign matter FM by using only the long-axis image of the foreign matter FM decided in Step S8. Here, the processing of Step S10 will be described in detail using the flowchart illustrated in FIG. 7. As illustrated in FIG. 7, Step S10 includes Step S13 to Step S18.

First, in Step S13, the foreign matter removal route guide unit 12 provides guidance that the removal route of the foreign matter FM is set, to the user. Although not illustrated, for example, the foreign matter removal route guide unit 12 displays the text, image, and the like representing that the removal route of the foreign matter FM is set on the display unit 7 to provide guidance to the user.

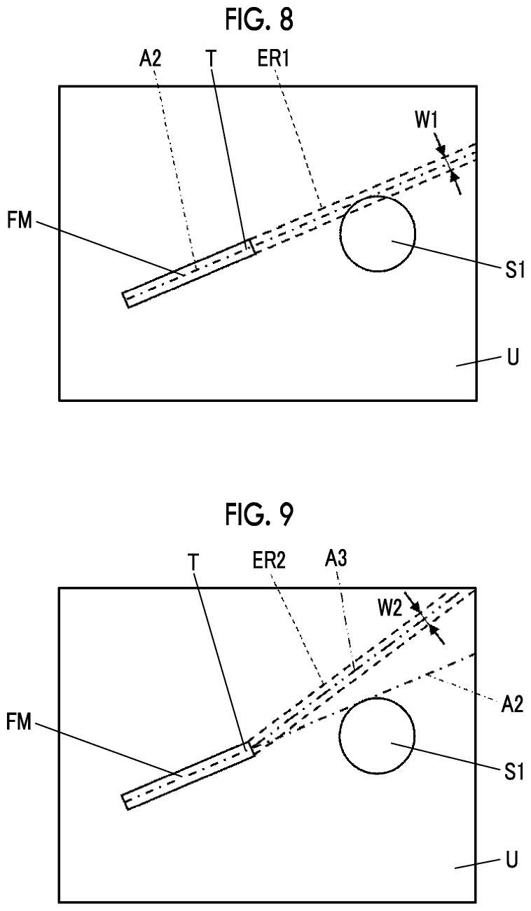

In Step S14, the foreign matter removal route setting unit 11 sets the removal route extending along a long axis A2 of the foreign matter FM as illustrated in FIG. 8, by using the long-axis image of the foreign matter FM obtained in Step S8. More specifically, the foreign matter removal route setting unit 11 sets an extension region ER1 where the foreign matter FM extends along the long axis A2 as the removal route of the foreign matter FM. The extension region ER1 extends from a distal end portion T of the foreign matter FM to the upper side of the ultrasound image U, that is, toward the body surface of the subject, and has a width W1 corresponding to the width of the foreign matter FM.

In Step S15, the delineation target detection unit 9 performs an image analysis on the long-axis image of the foreign matter FM to perform processing of detecting a peripheral tissue S1 consisting of blood vessels or nerves positioned around the foreign matter FM, as illustrated in FIG. 8.

In Step S16, the foreign matter removal route setting unit 11 determines whether the peripheral tissue S1 is detected in Step S15. In a case where it is determined in Step S16 that the peripheral tissue S1 is detected, the processing proceeds to Step S17.

In Step S17, the foreign matter removal route setting unit 11 determines whether the removal route set in Step S14 and the peripheral tissue S1 detected in Step S15 are in contact with each other on the basis of the long-axis image of the foreign matter FM. For example, in a case where the extension region ER1 as the removal route and the peripheral tissue S1 are in contact with each other as illustrated in FIG. 8, and it is determined in Step S17 that the removal route and the peripheral tissue S1 are in contact with each other, the processing proceeds to Step S18.

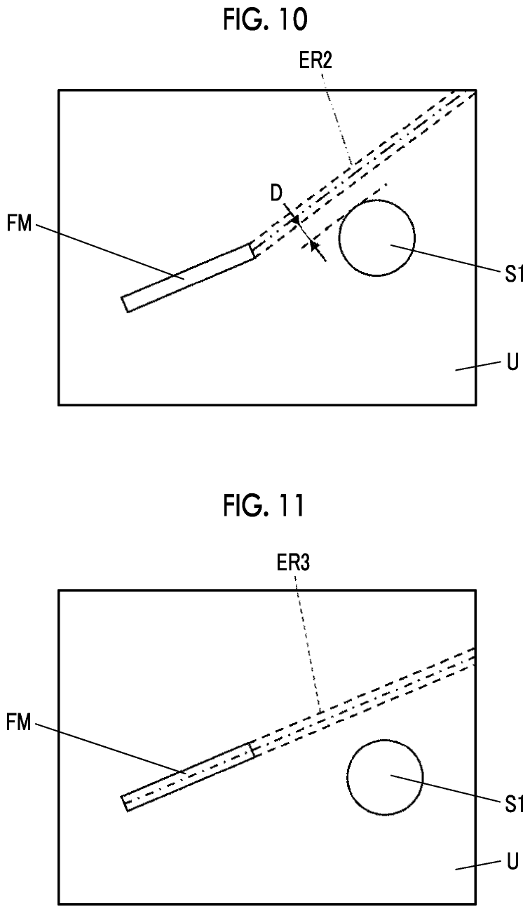

In Step S18, the foreign matter removal route setting unit 11 modifies the removal route set in Step S14 such that the removal route is not in contact with the peripheral tissue S1. In this case, as illustrated in FIG. 9, the foreign matter removal route setting unit 11 sets an inclination region ER2 which passes through the distal end portion T of the foreign matter FM and is obtained by tilting the extension region ER1, as the removal route of the foreign matter FM. The inclination region ER2 has a central axis A3 which is inclined to the upper side of the ultrasound image U, that is, toward the body surface of the subject with respect to the long axis A2 of the foreign matter FM, and has the same width W2 as the width W1 of the extension region ER1. For example, as illustrated in FIG. 10, the foreign matter removal route setting unit 11 can set the removal route such that the inclination region ER2 and the peripheral tissue S1 are apart from each other by a constant distance D. Here, the distance D can be set in a range of 4 mm to 5 mm, for example.

In a case where the removal route of the foreign matter FM is decided, the processing of Step S10 is completed.

Further, in a case where it is determined in Step S16 that the peripheral tissue S1 is not detected, the extension region set in Step S14 is decided as it is as the removal route, and the processing of Step S10 is completed.

Further, even in a case where it is determined in Step S17 that an extension region ER3 set as the removal route in Step S14 and the peripheral tissue S1 are not in contact with each other as illustrated in FIG. 11, the extension region ER3 is decided as it is as the removal route, and the processing of Step S10 is completed.

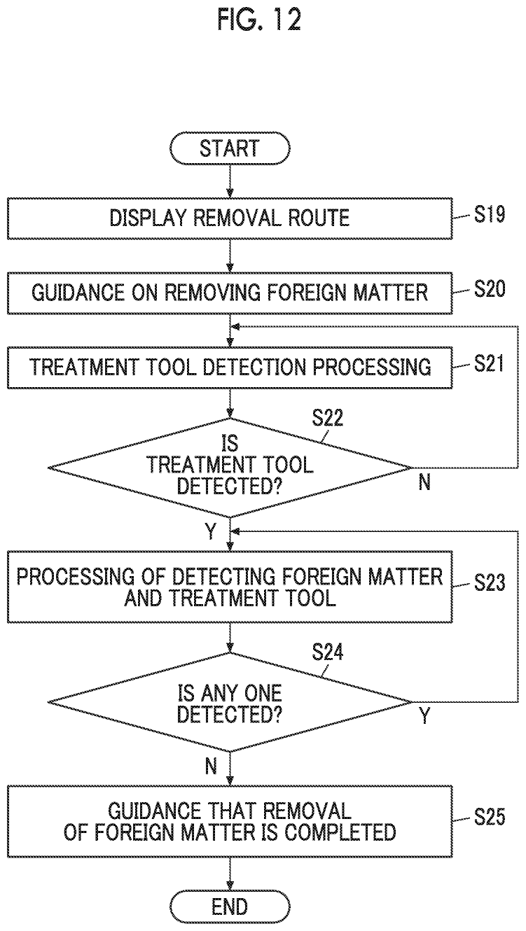

In a case where the processing in Step S10 is completed in this manner, the processing proceeds to Step S12. In Step S12, the foreign matter removal route guide unit 12 provides guidance on the removal route of the foreign matter FM set in Step S10, to the user. Here, the processing of Step S12 will be described in detail using FIG. 12. As illustrated in FIG. 12, Step S12 includes Step S19 to Step S25.

First, in Step S19, the foreign matter removal route guide unit 12 superimposes and displays the removal route of the foreign matter FM set in Step S10, on the ultrasound image U displayed on the display unit 7.

In Step S20, the foreign matter removal route guide unit 12 provides guidance that the foreign matter FM is removed, to the user. For example, although not illustrated, the foreign matter removal route guide unit 12 displays the text, image, and the like representing that the foreign matter FM is removed on the display unit 7 to provide guidance to the user. Accordingly, the user inserts the treatment tool for removing the foreign matter FM into the subcutaneous portion of the subject, and start the removal operation of the foreign matter FM.

In subsequent Step S21, the delineation target detection unit 9 performs an image analysis on the ultrasound image U to perform processing of detecting the treatment tool included in the ultrasound image U.

In Step S22, the foreign matter removal route guide unit 12 determines whether the treatment tool is detected in Step S21. In a case where it is determined in Step S22 that the treatment tool is not detected, the processing returns to Step S21, and the delineation target detection unit 9 newly performs processing of detecting the treatment tool. In this manner, Step S21 and Step S22 are repeated until it is determined in Step S22 that the treatment tool is detected.

In a case where it is determined in Step S22 that the treatment tool is detected, it is determined that the treatment tool is inserted into the subcutaneous portion of the subject, and the processing proceeds to Step S23. In Step S23, the delineation target detection unit 9 performs an image analysis on the ultrasound image U, and performs processing of detecting both the foreign matter FM and the treatment tool included in the ultrasound image U.

Here, in a state where the removal of the foreign matter FM is not completed, at least one of the foreign matter FM or the treatment tool is present in the subcutaneous portion of the subject, and therefore, at least one of the foreign matter FM or the treatment tool is detected in Step S23. On the other hand, in a state where the removal of the foreign matter FM is completed, both the foreign matter FM and the treatment tool are present outside the subject, and therefore, neither the foreign matter FM nor the treatment tool is detected in Step S23. Therefore, in Step S24, the foreign matter removal route guide unit 12 determines whether any one of the foreign matter FM and the treatment tool is detected in Step S23 in order to determine whether the removal of the foreign matter FM is completed. In a case where it is determined in Step S24 that at least one of the foreign matter FM or the treatment tool is detected, the processing returns to Step S23, and the delineation target detection unit 9 newly performs processing of detecting the foreign matter FM and the treatment tool. In this manner, processing of Step S23 and Step S24 is repeated until it is determined in Step S24 that any one of the foreign matter FM and the treatment tool is not detected.

Here, the emphasizing unit 14 can emphasize the foreign matter FM and the treatment tool detected in Step S23 and superimpose and display the resultant in the ultrasound image U displayed on the display unit 7. In this manner, the user can easily grasp the positions of the foreign matter FM and the treatment tool by visually observing the ultrasound image U, and therefore can more accurately perform the operation of the treatment tool.

In a case where it is determined in Step S24 that any one of the foreign matter FM and the treatment tool is not detected, it is determined that the foreign matter FM is removed from the subcutaneous portion of the subject by the treatment tool, and the processing proceeds to Step S25. In Step S25, the foreign matter removal route guide unit 12 provides guidance that the removal of the foreign matter FM is completed, to the user. For example, although not illustrated, the foreign matter removal route guide unit 12 displays the text, image, and the like representing that the removal of the foreign matter is completed on the display unit 7 to provide guidance to the user.

With the foreign matter removal route guide unit 12, in a case where the removal of the foreign matter FM is performed by the user, the removal route of the foreign matter FM which is set to avoid the peripheral tissue S1 is guided to the user, and therefore, the user can easily remove the foreign matter FM regardless of the proficiency.

In this manner, the processing of Step S12 is completed, and the operation of the ultrasound diagnostic apparatus 1 is ended.

In Step S9 of FIG. 4, in a case where the length of the foreign matter FM along the short axis, which is measured in Step S6, is greater than the predetermined threshold value, the processing proceeds to Step S11.

In Step S11, the foreign matter removal route setting unit 11 sets the removal route of the foreign matter FM by using the ultrasound images U of the plurality of frames including the foreign matter FM, in order to set the removal route in which the foreign matter FM and the peripheral tissue are not in contact with each other in the depth direction of the long-axis image of the foreign matter FM displayed in Step S8, that is, in the direction parallel to the short axis of the foreign matter FM.

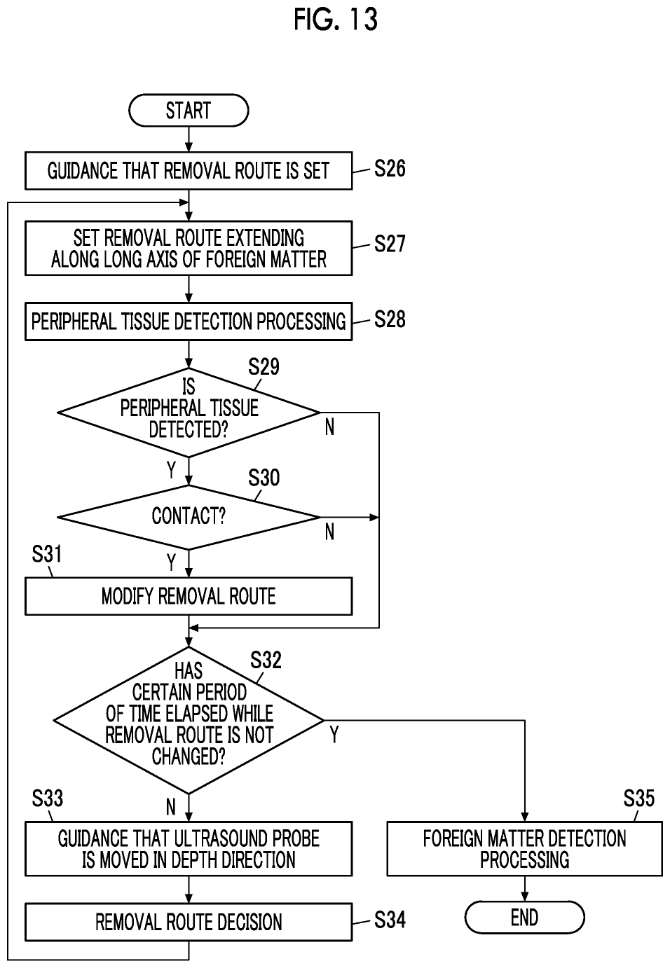

Here, the processing of Step S11 will be described in detail using the flowchart illustrated in FIG. 13. The flowchart illustrated in FIG. 13 includes Step S26 to Step S35, and Step S26 to Step S31 are the same as Step S13 to Step S18 illustrated in FIG. 7.

First, in Step S26, the foreign matter removal route guide unit 12 provides guidance that the removal route of the foreign matter FM is set, to the user.

Next, in Step S27, the foreign matter removal route setting unit 11 sets, as the removal route of the foreign matter FM, the extension region ER1 extending along the long axis A2 of the foreign matter FM as illustrated in FIG. 8, by using the long-axis image of the foreign matter FM obtained in Step S8.

In Step S28, the delineation target detection unit 9 performs an image analysis on the long-axis image of the foreign matter FM to perform processing of detecting the peripheral tissue S1 consisting of blood vessels or nerves positioned around the foreign matter FM, as illustrated in FIG. 8.

In Step S29, the foreign matter removal route setting unit 11 determines whether the peripheral tissue S1 is detected in Step S28. In a case where it is determined in Step S29 that the peripheral tissue S1 is detected, the processing proceeds to Step S30.

In Step S30, the foreign matter removal route setting unit 11 determines whether the removal route set in Step S27 and the peripheral tissue S1 detected in Step S28 are in contact with each other on the basis of the long-axis image of the foreign matter FM. In a case where it is determined in Step S30 that the removal route and the peripheral tissue S1 are in contact with each other, the processing proceeds to Step S31.

In Step S31, the foreign matter removal route setting unit 11 modifies the removal route set in Step S27 such that the removal route is not in contact with the peripheral tissue S1, as illustrated in FIG. 9, for example. In a case where the removal route is set in this manner, the processing proceeds to Step S32.

Here, in a case where it is determined in Step S29 that the peripheral tissue S1 is not detected, the extension region set in Step S27 is decided as it is as the removal route, and the processing proceeds to Step S32.

Further, even in a case where it is determined in Step S30 that the extension region ER3 set as the removal route in Step S27 and the peripheral tissue S1 are not in contact with each other as illustrated in FIG. 11, the extension region ER3 is decided as it is as the removal route, and the processing proceeds to Step S32.

In Step S32, the foreign matter removal route setting unit 11 determines whether a certain period of time has elapsed in a state where the removal route is not changed. Here, the change of the removal route means that a new removal route which is different from the removal route already set by performing the processing of Step S27 to Step S31 on the ultrasound images U of the plurality of frames is set. In a case where the removal route of the foreign matter FM is set for the first time in Step S27 to Step S31, since the removal route is not changed, and a certain period of time has not elapsed, it is determined in Step S32 that a certain period of time has not elapsed in a state where the removal route is not changed, and the processing proceeds to Step S33. The certain period of time is preferably set in a range of 3 seconds to 5 seconds.

In Step S33, the probe operation guide unit 13 provides, to the user, guidance that the ultrasound probe 18 is moved in the depth direction of the long-axis image of the foreign matter FM so that the foreign matter is delineated by the plurality of tomographic images along the long axis of the foreign matter FM. In this manner, the user moves the ultrasound probe 18 according to the guidance by the probe operation guide unit 13, and the plurality of tomographic images along the long axis of the foreign matter FM are acquired by the image acquisition unit 8.

In Step S34, the delineation target detection unit 9 performs an image analysis on the ultrasound images acquired by the image acquisition unit 8, and detects the foreign matter FM included in the tomographic images along the long axis of the foreign matter FM. In a case where the processing of Step S34 is completed in this manner, the processing proceeds to Step S27.

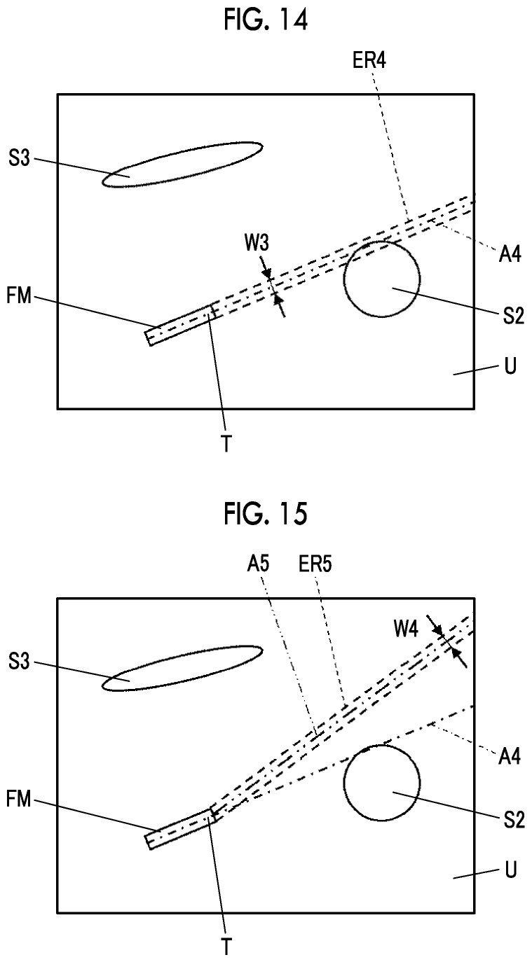

In Step S27, the foreign matter removal route setting unit 11 sets, as the removal route of the foreign matter FM, an extension region ER4 extending along a long axis A4 of the foreign matter FM as illustrated in FIG. 14, on the basis of the ultrasound image U newly acquired by the image acquisition unit 8 in a state where the ultrasound probe 18 is moved by the user according to the guidance in Step S33. Here, the long axis A4 of the foreign matter FM is the same as the long axis A2 of the foreign matter FM detected in Step S8 in the flowchart illustrated in FIG. 4. In the example illustrated in FIG. 14, the extension region ER4 has a width W3 corresponding to the width of the foreign matter FM.

In subsequent Step S28, the delineation target detection unit 9 performs an image analysis on the ultrasound image U, and detects the peripheral tissue included in the ultrasound image U. Here, by the user moving the ultrasound probe 18 according to the guidance in Step S33, a plurality of peripheral tissues present in the depth direction with respect to the long-axis image of the foreign matter FM are included in the ultrasound image U. In the example illustrated in FIG. 14, two peripheral tissues S2 and S3 are included in the ultrasound image U, and the peripheral tissues S2 and S3 are detected by the delineation target detection unit 9.

In Step S29, the foreign matter removal route setting unit 11 determines whether the peripheral tissues are detected in Step S28. In a case where it is determined in Step S29 that the peripheral tissues S2 and S3 are detected, the processing proceeds to Step S30.

In Step S30, the foreign matter removal route setting unit 11 determines whether the removal route or the foreign matter FM set in Step S27 and the peripheral tissues S2 and S3 detected in Step S28 are in contact with each other. For example, in a case where the extension region ER4 set as the removal route and the peripheral tissue S2 are in contact with each other as illustrated in FIG. 14, and it is determined in Step S30 that the removal route and the peripheral tissue S2 are in contact with each other, the processing proceeds to Step S31.

In Step S31, the foreign matter removal route setting unit 11 modifies the removal route set in Step S27 such that the removal route is not in contact with the peripheral tissues S2 and S3. In this case, as illustrated in FIG. 15, the foreign matter removal route setting unit 11 sets an inclination region ER5 which passes through the distal end portion T of the foreign matter FM and is obtained by tilting the extension region ER4, as the removal route of the foreign matter FM. The inclination region ER5 has a central axis A5 which is inclined toward the body surface of the subject with respect to the long axis A4 of the foreign matter FM, and has the same width W4 as the width W3 of the extension region ER4. For example, the foreign matter removal route setting unit 11 can set the removal route such that the inclination region ER5 is apart from each of the peripheral tissues S2 and S3 and the inclination region ER5 and the peripheral tissue S2 which is closest to the inclination region ER5 are apart from each other by the predetermined distance D. In a case where the removal route is modified in this manner, the processing proceeds to Step S32.

Here, in a case where it is determined in Step S29 that the peripheral tissues S2 and S3 are not detected, the extension region set in Step S27 is decided as it is as the removal route, and the processing proceeds to Step S32.

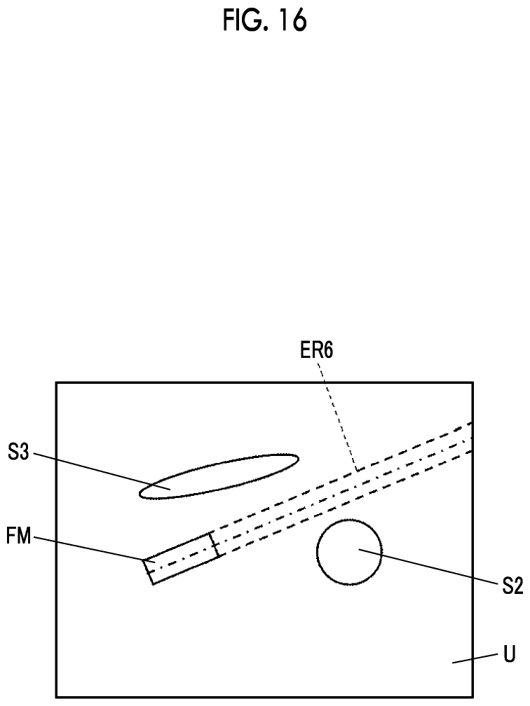

Further, even in a case where it is determined in Step S30 that an extension region ER6 set as the removal route in Step S27 and the peripheral tissues S2 and S3 are not in contact with each other as illustrated in FIG. 16, the extension region ER6 is decided as it is as the removal route, and the processing proceeds to Step S32.

In subsequent Step S32, the foreign matter removal route setting unit 11 determines whether a certain period of time has elapsed in a state where the removal route is not changed. In this manner, the processing of Step S27 to Step S34 is repeated until it is determined in Step S32 that a certain period of time has elapsed in a state where the removal route is not changed. In a case where it is determined in Step S32 that a certain period of time has elapsed in a state where the removal route is not changed, the processing proceeds to Step S35.

In Step S35, the foreign matter removal route setting unit 11 decides the removal route finally set in Step S27 as the removal route actually used for removing the foreign matter FM. In this manner, the processing of Step S11 is completed.

In this manner, in a case where the length of the foreign matter FM along the short axis is greater than the threshold value, the removal route is set to avoid the peripheral tissue present in the depth direction of the long-axis image of the foreign matter FM, and therefore, in a case where the foreign matter FM is removed, the foreign matter FM can be easily removed without damaging the peripheral tissue.

In subsequent Step S12, the foreign matter removal route guide unit 12 provides guidance on the removal route set in Step S11, to the user. In a case where the guidance in Step S11 is completed, the operation of the ultrasound diagnostic apparatus 1 is ended.

As described above, with the ultrasound diagnostic apparatus 1 according to the first embodiment of the invention, since the removal route of the foreign matter FM is automatically set to avoid the peripheral tissue positioned around the foreign matter FM by the foreign matter removal route setting unit 11, and the removal route is guided to the user by the foreign matter removal route guide unit 12, the user can easily remove the foreign matter FM regardless of the proficiency.

In the first embodiment, the probe operation guide unit 13 displays the text, image, and the like on the display unit 7 to provide the guidance on the operation of the ultrasound probe 18, to the user, but the method of providing guidance to the user is not limited thereto. For example, a sound generation unit (not illustrated) can be provided to the ultrasound diagnostic apparatus 1, and the probe operation guide unit 13 can provided guidance to the user by using the sound via the sound generation unit.

Further, in the first embodiment, in a case where the length of the foreign matter FM along the short axis is equal to or less than the threshold value, the removal route of the foreign matter FM is set by using only the long-axis image of the foreign matter FM, and in a case where the length of the foreign matter FM along the short axis is greater than the threshold value, the removal route of the foreign matter FM is set by using the ultrasound images of the plurality of frames, but the threshold value can be set by the user via the input unit 16, for example. For example, the threshold value can be set in a range of 1.5 times to 2 times the width of the treatment tool used for removing the foreign matter FM.

In a case where the position of the treatment tool detected by the delineation target detection unit 9 deviates from the removal route, that is, in a case where the treatment tool is position outside the removal route, the foreign matter removal route guide unit 12 can provide guidance that the treatment tool deviates from the removal route, to the user. In this case, for example, the foreign matter removal route guide unit 12 displays the text, image, and the like representing that the treatment tool deviates from the removal route on the display unit 7 to provide guidance to the user. Further, a sound generation unit (not illustrated) can be provided to the ultrasound diagnostic apparatus 1, and the foreign matter removal route guide unit 12 can provided guidance to the user by using the sound via the sound generation unit.

### Second Embodiment

In the first embodiment, the three-dimensional structure acquisition unit 10 of the processor 19 measures the length L1 along the long axis and the length L2 along the short axis of the foreign matter FM as the three-dimensional structure of the foreign matter FM, but a three-dimensional model (3D model) of the foreign matter FM may be constructed as the three-dimensional structure of the foreign matter FM.

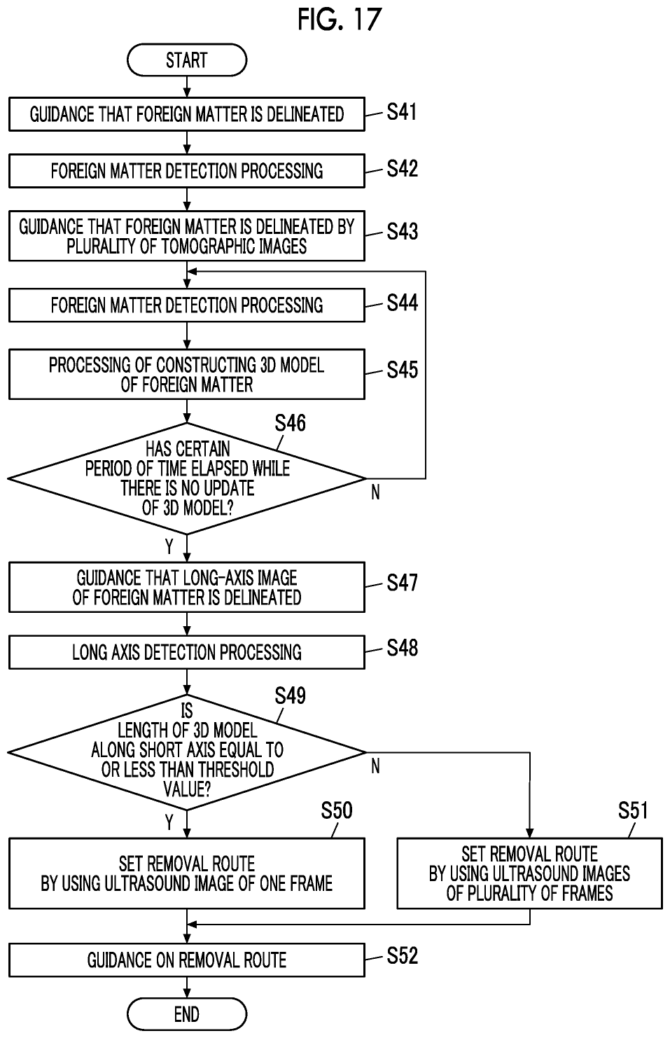

Next, the operation of the ultrasound diagnostic apparatus 1 in a second embodiment will be described in detail using the flowchart illustrated in FIG. 17. The flowchart of FIG. 17 includes Step S41 to Step S52, but Step S41, Step S42, Step S47, and Step S50 to Step S52 are the same as Step S1, Step S2, Step S7, and Step S10 to Step S12 in the flowchart of FIG. 4, respectively.

In Step S41, the probe operation guide unit 13 provides guidance that the foreign matter FM is delineated on the display unit 7, to the user. In this manner, the user moves the ultrasound probe 18 according to the guidance by the probe operation guide unit 13. Further, in this case, the image acquisition unit 8 acquires the ultrasound images U sequentially and continuously.

Next, in Step S42, the delineation target detection unit 9 performs an image analysis on the ultrasound images U acquired by the image acquisition unit 8, and performs processing of detecting the foreign matter FM as the delineation target. In this case, the delineation target detection unit 9 performs the processing of detecting the foreign matter FM on each of the ultrasound images U sequentially acquired by the image acquisition unit 8 until the foreign matter FM is detected. In a case where the foreign matter FM is detected in Step S42, the processing proceeds to Step S43.

In Step S43, the probe operation guide unit 13 provides guidance that the foreign matter FM is delineated by a plurality of tomographic images, to the user. In this case, for example, the probe operation guide unit 13 displays the text, image, and the like representing that the foreign matter FM is delineated by the plurality of tomographic images on the display unit 7 to provide guidance to the user. In this manner, the user moves the ultrasound probe 18 according to the guidance by the probe operation guide unit 13. Further, in this case, the image acquisition unit 8 acquires the ultrasound images U sequentially and continuously.

In Step S44, the delineation target detection unit 9 performs processing of detecting the foreign matter FM on each of the ultrasound images U of the plurality of frames sequentially acquired by the image acquisition unit 8, as in Step S42.

In Step S45, the three-dimensional structure acquisition unit 10 constructs the 3D model of the foreign matter FM by synthesizing the image of the foreign matter FM detected in Step S44 with the ultrasound images U of the plurality of frames representing the plurality of tomographic images which are acquired by the image acquisition unit 8.

In Step S46, the three-dimensional structure acquisition unit 10 determines whether a certain period of time has elapsed in a state where there is no update of the 3D model. Here, the update of the 3D model means that the 3D model already constructed by performing the processing of Step S44 and Step S45 on the ultrasound images U of the plurality of frames is updated to a more accurate 3D model. In a case where the 3D model of the foreign matter FM is constructed for the first time in Step S45, since the 3D model is not updated, and a certain period of time has not elapsed, it is determined in Step S46 that a certain period of time has not elapsed in a state where there is no update of the 3D model, and the processing returns to Step S44. Here, the certain period of time is preferably set in a range of 3 seconds to 5 seconds.

In Step S44, the delineation target detection unit 9 newly detects the foreign matter FM, and in Step S45, the three-dimensional structure acquisition unit 10 performs processing of constructing the 3D model on the basis of the image of the foreign matter FM detected in Step S44. In this manner, in a case where the accuracy of the 3D model is improved by repeating the processing of Step S44 to Step S46 so that there is no more update of the 3D model and a certain period of time has elapsed, it is determined in Step S46 that a certain period of time has elapsed in a state where there is no update of the 3D model, and the processing proceeds to Step S47.

In Step S47, the probe operation guide unit 13 provides guidance that the long-axis image of the foreign matter FM is delineated on the display unit 7, to the user. In this manner, the user moves the ultrasound probe 18 according to the guidance by the probe operation guide unit 13.

In Step S48, the three-dimensional structure acquisition unit 10 performs an image analysis on each of the ultrasound images U of the plurality of frames sequentially acquired by the image acquisition unit 8, and performs processing of detecting the long axis of the foreign matter FM included in the ultrasound image U. In this case, for example, as illustrated in FIG. 5, first, the three-dimensional structure acquisition unit 10 detects the principal axis of inertia A1 of the foreign matter FM in the ultrasound image U, and measures the length L1 of the foreign matter FM along the detected principal axis of inertia A1. In this case, the three-dimensional structure acquisition unit 10 performs the detection of the principal axis of inertia A1 and the measurement of the length L1 on each of the ultrasound images U sequentially acquired by the image acquisition unit 8. Further, for example, in a case where the measured length L1 is greater than a value obtained by multiplying the length along the long axis of the 3D model constructed in Step S45 by a constant coefficient, the three-dimensional structure acquisition unit 10 decides the principal axis of inertia A1 in this case as the long axis of the foreign matter FM, and decides the ultrasound image U including the principal axis of inertia A1 as the long-axis image of the foreign matter FM. Here, the coefficient by which the length along the long axis of the 3D model is multiplied is preferably set in a range of 0.9 to 1.0, and is set to 0.9, for example.

In subsequent Step S49, the foreign matter removal route setting unit 11 determines whether the length along the short axis of the 3D model is equal to or less than a predetermined threshold value. In a case where it is determined in Step S49 that the length along the short axis of the 3D model is equal to or less than the threshold value, the processing proceeds to Step S50.

In Step S50, the foreign matter removal route setting unit 11 sets the removal route of the foreign matter FM by using the ultrasound image U of one frame which is decided as the long-axis image of the foreign matter FM in Step S48. More specifically, the foreign matter removal route setting unit 11 sets the removal route of the foreign matter FM according to Step S13 to Step S18 in the flowchart of FIG. 7.

In a case where the removal route of the foreign matter FM is set in Step S50, the processing proceeds to Step S52. In Step S52, the foreign matter removal route guide unit 12 provides guidance on the removal route of the foreign matter FM set in Step S50, to the user. More specifically, the foreign matter removal route guide unit 12 provides guidance on the removal route, to the user, according to Step S19 to Step S25 in the flowchart of FIG. 12. In a case where the processing of Step S52 is performed in this manner, the operation of the ultrasound diagnostic apparatus 1 is ended.

Further, in a case where it is determined in Step S49 that the length along the short axis of the 3D model is greater than the threshold value, the processing proceeds to Step S51.

In Step S51, the foreign matter removal route setting unit 11 sets the removal route of the foreign matter by using the ultrasound images of the plurality of frames representing tomographic planes parallel to the long axis of the foreign matter FM detected in Step S48. More specifically, the foreign matter removal route setting unit 11 sets the removal route of the foreign matter FM according to Step S26 to Step S35 in the flowchart of FIG. 13.

In a case where the removal route of the foreign matter FM is set in Step S51, the processing proceeds to Step S52. In Step S52, the foreign matter removal route guide unit 12 provides guidance on the removal route of the foreign matter FM set in Step S51, to the user. In a case where the processing of Step S52 is performed in this manner, the operation of the ultrasound diagnostic apparatus 1 is ended.

As described above, according to the second embodiment, even in a case where the 3D model of the foreign matter FM is constructed as the three-dimensional structure of the foreign matter FM by the three-dimensional structure acquisition unit 10, since the removal route of the foreign matter FM is automatically set to avoid the peripheral tissue positioned around the foreign matter FM by the foreign matter removal route setting unit 11, and the removal route is guided to the user by the foreign matter removal route guide unit 12 as in the first embodiment, the user can easily remove the foreign matter FM regardless of the proficiency.

### EXPLANATION OF REFERENCES

1: ultrasound diagnostic apparatus
2: transducer array
3: transmission unit
4: reception unit
5: image generation unit
6: display controller
7: display unit
8: image acquisition unit
9: delineation target detection unit
10: three-dimensional structure acquisition unit
11: foreign matter removal route setting unit
12: foreign matter removal route guide unit
13: probe operation guide unit
14: emphasizing unit
15: device controller
16: input unit
17: storage unit
18: ultrasound probe
19: processor
20: amplification unit
21: AD conversion unit
22: signal processing unit
23: DSC
24: image processing unit
A1: principal axis of inertia
A2, A4: long axis
A3, A5: central axis
D: distance
ER1, ER3, ER4, ER6: extension region
ER2, ER5: inclination region
FM: foreign matter
L1, L2: length
P1, P2: measured value panel
S1, S2, S3: peripheral tissue
T: distal end portion
U: ultrasound image
W1, W2, W3, W4: width

What is claimed is:

1. An ultrasound diagnostic apparatus comprising:
an ultrasound probe;
a display unit; and
a processor configured to
perform transmission of an ultrasound beam from the ultrasound probe toward a subject to acquire an ultrasound image,
perform an image analysis on the acquired ultrasound image to detect a foreign matter entered a subcutaneous portion of the subject and a peripheral tissue of the subject consisting of blood vessels or nerves positioned around the foreign matter,
automatically set a removal route of the foreign matter to avoid the peripheral tissue on the basis of the detected foreign matter and the detected peripheral tissue, and
display the set removal route on the display unit.

2. The ultrasound diagnostic apparatus according to claim 1,
wherein the processor displays the removal route on the display unit by superimposing and displaying the removal route on the ultrasound image displayed on the display unit.

3. The ultrasound diagnostic apparatus according to claim 2,
wherein the processor is configured to
provide, to a user, guidance on an operation of the ultrasound probe such that the foreign matter is delineated on the display unit by a plurality of tomographic images,
acquire a three-dimensional structure of the foreign matter on the basis of ultrasound images of a plurality of frames representing the plurality of tomographic images of the foreign matter, and
set the removal route on the basis of a position of the foreign matter, the three-dimensional structure of the foreign matter, and a position of the peripheral tissue.

4. The ultrasound diagnostic apparatus according to claim 3,
wherein the processor is configured to
display the operation of the ultrasound probe on the display unit such that the foreign matter is delineated by a first tomographic image along a long axis of the foreign matter and a second tomographic image along a short axis of the foreign matter on the display unit,
acquire a length along the short axis of the foreign matter as the three-dimensional structure of the foreign matter from the ultrasound image representing the first tomographic image and the ultrasound image representing the second tomographic image, and
set the removal route on the basis of the position of the foreign matter, the length along the short axis of the foreign matter, and the position of the peripheral tissue.

5. The ultrasound diagnostic apparatus according to claim 4,
wherein in a case where the length along the short axis of the foreign matter acquired as the three-dimensional structure of the foreign matter is equal to or less than a predetermined threshold value, the processor sets the removal route such that the foreign matter avoids the peripheral tissue in the first tomographic image.

6. The ultrasound diagnostic apparatus according to claim 5,
wherein in a case where an extension region where the foreign matter extends along a long axis direction is apart from the peripheral tissue in the first tomographic image, the processor sets the extension region as the removal route, and in a case where the extension region is in contact with the peripheral tissue in the first tomographic image, the processor sets an inclination region which passes through a distal end portion of the foreign matter and is obtained by tilting the extension region to avoid the peripheral tissue, as the removal route.

7. The ultrasound diagnostic apparatus according to claim 4,
wherein in a case where the length along the short axis of the foreign matter acquired as the three-dimensional structure of the foreign matter is greater than a predetermined threshold value, the processor displays the operation of the ultrasound probe on the display unit such that the foreign matter is delineated by a plurality of tomographic images along a long axis of the foreign matter on the display unit, and the processor sets the removal route such that the foreign matter avoids the peripheral tissue in the plurality of tomographic images.

8. The ultrasound diagnostic apparatus according to claim 7,
wherein in a case where an extension region where the foreign matter extends along a long axis direction is apart from the peripheral tissue in the plurality of tomographic images, the processor sets the extension region as the removal route, and in a case where the extension region is in contact with the peripheral tissue in the plurality of tomographic images, the processor sets an inclination region which passes through a distal end portion of the foreign matter and is obtained by tilting the extension region to avoid the peripheral tissue, as the removal route.

9. The ultrasound diagnostic apparatus according to claim 3,
wherein the processor is configured to
acquire a three-dimensional model of the foreign matter by synthesizing an image of the foreign matter with the plurality of ultrasound images representing the plurality of tomographic images, and
set the removal route on the basis of the position of the foreign matter, a length along a short axis of the three-dimensional model, and the position of the peripheral tissue.

10. The ultrasound diagnostic apparatus according to claim 9,
wherein in a case where the length along the short axis of the three-dimensional model acquired as the three-dimensional structure of the foreign matter is equal to or less than a predetermined threshold value, the processor sets the removal route such that the foreign matter avoids the peripheral tissue in the tomographic image along a long axis of the foreign matter.

11. The ultrasound diagnostic apparatus according to claim 10,
wherein in a case where an extension region where the foreign matter extends along a long axis direction is apart from the peripheral tissue in the tomographic image along the long axis of the foreign matter, the processor sets the extension region as the removal route, and in a case where the extension region is in contact with the peripheral tissue in the tomographic image along the long axis of the foreign matter, the processor sets an inclination region which passes through a distal end portion of the foreign matter and is obtained by tilting the extension region to avoid the peripheral tissue, as the removal route.

12. The ultrasound diagnostic apparatus according to claim 9,
wherein in a case where the length along the short axis of the three-dimensional model acquired as the three-dimensional structure of the foreign matter is greater than a predetermined threshold value, the processor displays the operation of the ultrasound probe on the display unit such that the foreign matter is delineated by a plurality of tomographic images along a long axis of the foreign matter on the display unit, and the processor sets the removal route such that the foreign matter avoids the peripheral tissue in the plurality of tomographic images.

13. The ultrasound diagnostic apparatus according to claim 12,
wherein in a case where an extension region where the foreign matter extends along a long axis direction is apart from the peripheral tissue in the plurality of tomographic images, the processor sets the extension region as the removal route, and in a case where the extension region is in contact with the peripheral tissue in the plurality of tomographic images, the processor sets an inclination region which passes through a distal end portion of the foreign matter and is obtained by tilting the extension region to avoid the peripheral tissue, as the removal route.

14. The ultrasound diagnostic apparatus according to claim 2,
wherein the processor is configured to
detect a treatment tool for removing the foreign matter, and
provide guidance to a user to modify a position of the treatment tool in a case where the detected treatment tool deviates from the removal route.

15. The ultrasound diagnostic apparatus according to claim 3,
wherein the processor is configured to
detect a treatment tool for removing the foreign matter, and
provide guidance to a user to modify a position of the treatment tool in a case where the detected treatment tool deviates from the removal route.

16. The ultrasound diagnostic apparatus according to claim 4,
wherein the processor is configured to
detect a treatment tool for removing the foreign matter, and
provide guidance to a user to modify a position of the treatment tool in a case where the detected treatment tool deviates from the removal route.

17. The ultrasound diagnostic apparatus according to claim 5,
wherein the processor is configured to
detect a treatment tool for removing the foreign matter, and
provide guidance to a user to modify a position of the treatment tool in a case where the detected treatment tool deviates from the removal route.

18. The ultrasound diagnostic apparatus according to claim 7,
wherein the processor is configured to
detect a treatment tool for removing the foreign matter, and
provide guidance to a user to modify a position of the treatment tool in a case where the detected treatment tool deviates from the removal route.

19. The ultrasound diagnostic apparatus according to claim 14,
wherein the processor is configured to emphasize at least one of the detected foreign matter or the detected treatment tool and display the resultant on the display unit.

20. A control method of an ultrasound diagnostic apparatus, the control method comprising:
performing transmission of an ultrasound beam toward a subject to acquire an ultrasound image;
performing an image analysis on the acquired ultrasound image to detect a foreign matter entered a subcutaneous portion of the subject and a peripheral tissue of the subject consisting of blood vessels or nerves positioned around the foreign matter;
automatically setting a removal route of the foreign matter to avoid the peripheral tissue on the basis of the detected foreign matter and the detected peripheral tissue; and
displaying the set removal route on a display unit.
